(19) United States
(12) Patent Application Publication
(10) Pub. No.: US 2019/0284282 A1
(43) Pub. Date: Sep. 19, 2019

(54) STABLE PHARMACEUTICAL COMPOSITION

(71) Applicant: DR. REDDY'S LABORATORIES LIMITED, Hyderabad (IN)

(72) Inventors: Murali JAYARAMAN, Kancheepuram (IN); Pravin NAIR, Mumbai (IN); Lakshmi KANAKADURGA, Hyderabad (IN); Navneet KAUR, Ludhiana (IN); Deepak THUMBRAHALLI, Kampli (IN)

(73) Assignee: Dr. Reddy's Laboratories Limited, Hyderabad (IN)

(21) Appl. No.: 16/068,294

(22) PCT Filed: Jan. 11, 2017
(86) PCT No.: PCT/IB2017/050116

§ 371 Date: Jul. 5, 2018

(30) Foreign Application Priority Data
Jan. 12, 2016 (IN) 201641001111

(51) Int. Cl. C07K 16/28; A61K 9/08; A61K 47/02; A61K 47/12; A61K 47/18; A61K 47/26

(52) U.S. Cl. CPC C07K 16/2866; A61K 9/08; A61K 47/26; A61K 47/12; A61K 47/183; A61K 47/02

(57) ABSTRACT

This present invention relates to a method of stabilizing an aqueous pharmaceutical preparation susceptible to de-gradation by formulating in a dual buffer system wherein the individual buffers are selected from phosphate, aspartate, glutamate, and succinate buffer.

### RELATED APPLICATION

This application is related to and takes priority from Indian Provisional Application 201641001111 filed 12 Jan. 2016 and is herein incorporated in its entirety.

### BACKGROUND OF THE INVENTION

Advances in biotechnology have paved way for the possibility of producing a variety of proteins for pharmaceutical applications. However, proteins are larger and more complex three-dimensional structures comprising multiple functional groups than traditional drugs. Proteins are generally known to be unstable in solution and sensitive to pH, temperature and oxidation and hence can undergo a variety of covalent and non-covalent reactions, modifications or degradations in solution.

The more common protein degradation pathways include aggregation, deamidation and oxidation. These pathways lead to both physical and chemical instability of a protein in solution. Chemical instability can be a result of deamidation, hydrolysis, oxidation or disulfide exchange whereas physical instability can be a result of denaturation, aggregation, adsorption or precipitation.

Protein aggregation is of particular interest in protein formulation because it often results in diminished bioactivity of the protein that affects drug potency, and may also elicit serious immunological reactions in patients. Chemical degradation of a protein therapeutic, has also been implicated in increasing its antigenic potential. Thus proteins pose inherent challenges to formulation for therapeutic uses.

Hence, stability of a protein formulation is one of the most important criteria for ensuring safety and consistent and effective administration. Any loss in bioactivity of the protein within a composition will reduce its effective concentration. Similarly any undesirable protein modifications may lead to loss of efficacy, as well increase the risk of adverse events. Hence, a stable composition requires having proteins formulated in an appropriate buffer that ensures stability vis a vis protein degradation pathways.

The state of the art lists the use of excipients in formulations to prevent aggregation, denaturation or similar other degradations. Sugars such as sucrose, glucose, raffinose and trehalose, and polyols such as glycerol, sorbitol and mannitol have been used as protein stabilizers. The concentration of sugars and polyols in any protein composition is directly proportional to the stability of the protein. (Foster et al, Int. J. Pharm. (1996) 134(1,2): 193-201). Other excipients used in protein formulation include use of amino acids, amino sugars, salts and polaxamers etc.

The choice of excipients while formulating a protein is governed by various factors including, their compatibility with the protein, as well as other components in the formulation, mode of administration, dosage, therapeutic indication etc. Therefore rational behind a formulation development involves screening and selection of suitable buffer conditions and excipients, as well as their concentrations.

The present invention addresses the challenges in the art by developing a stable pharmaceutical formulation that maintain protein solubility, stability and bioactivity of the active ingredient.

### SUMMARY OF THE INVENTION

The present invention discloses a stable aqueous formulation for an antibody comprising a dual buffer system wherein the individual buffers are selected from phosphate, aspartate, glutamate, and succinate. The disclosed antibody formulation provides improved stability for extended period of time even under accelerated stability conditions. The stable formulation inhibits the formation of undesirable antibody variants and thus maintains the physical, chemical or biological properties of the antibody composition. Furthermore, the formulation so disclosed can be used for a number of different therapeutic antibodies.

### BRIEF DESCRIPTION OF DRAWINGS

FIG. 1: Illustrates osmolality shift for A-mab formulation at T0 and T4W at 37° C. as disclosed in example-1.

FIG. 2: Illustrates trends in % high molecular weight species (HMWS) in SEC over time under stressed conditions (50° C. for 2 weeks) for A-mab composition.

FIG. 3: Illustrates trends in % of monomer loss in SEC over time under stressed conditions (50° C. for 2 weeks) for A-mab composition.

FIG. 4: Illustrates trends in IEX % of basic variants over time under stressed conditions (50° C. for 2 weeks) for A-mab composition.

FIG. 5: Illustrates trends in Light scattering using nanodrop over time under stressed conditions (50° C. for 2 weeks). Here, T0 represents data at day ‘0’ and T1W represents one week data at 50° C. for A-mab composition.

### DETAILED DESCRIPTION OF THE INVENTION

The present invention discloses a stable aqueous pharmaceutical formulation for an antibody wherein the antibody is formulated in a dual buffer system.

In one embodiment of the invention, the dual buffer system comprises the combination of any of the two buffers selected from a group consisting of phosphate, aspartate, succinate and glutamate.

In another embodiment of the invention, the dual buffer system consists of phosphate buffer and glutamate buffer.

An embodiment of the invention discloses a stable aqueous pharmaceutical formulation for an antibody wherein the antibody is formulated in a dual buffer system, and which further comprises pharmaceutically acceptable excipients.

In any of the above mentioned embodiments the antibody formulated in a dual buffer system is stable at 2-8° C. for at least 2 years.

In any of the above mentioned embodiments of the invention, the antibody formulated in a dual buffer system is stable at 25° C. for at least 3 months.

In any of the above mentioned embodiments of the invention, the antibody formulated in a dual buffer system is stable at about 40° C. for at least 2 weeks, more preferably for at least 4 weeks.

In any of the above mentioned embodiments of the invention, the antibody formulated in a dual buffer system is stable at about 50° C. for at least 1 week, more preferably for at least 2 weeks.

In any of the above mentioned embodiments of the invention, the antibody formulated in dual buffer system is stable following three freeze-thaw cycles, preferably five freeze-thaw cycles.

In another embodiment, the invention discloses an antibody formulation comprising excipients wherein the excipients comprise amino acids, preferably the amino acids are arginine and/or glycine or derivatives and their combination thereof.

In yet another embodiment, the invention discloses an antibody formulation comprising excipients wherein the excipients comprise sugars or sugar alcohol, preferably the sugars are mannitol, sorbitol, sucrose and trehalose or derivatives and their combination thereof.

In a further embodiment, the invention discloses an antibody formulation comprising excipients wherein the excipients comprise surfactant, preferably the surfactant is polysorbate 80.

In another embodiment, the invention discloses an antibody formulation comprising excipients wherein the excipients comprise salts, more preferably the salt is sodium chloride.

In any of the above mentioned embodiments of the invention, the pH of the dual buffer system is from about 5 to about 7, more preferably the pH of the dual buffer system is about 5.2 to about 6.0.

In any of the above mentioned embodiments of the invention, the antibody is present at a concentration of at least 20 mg/ml in the formulation, more preferably at least 50 mg/ml and further more preferably at least 100 mg/ml.

In any of the above mentioned embodiments of the invention, the antibody in the formulation is a therapeutic antibody.

In any of the above mentioned embodiments, the antibody in the formulation is selected from an anti-TNFα antibody, an anti-IL-6R antibody, an anti-HER2 antibody, more preferably selected from a group consisting of adalimumab, tocilizumab or trastuzumab.

In an embodiment, the invention discloses an aqueous pharmaceutical formulation of a therapeutic antibody comprising a dual buffer system, wherein individual buffer in the dual buffer system are selected from a group consisting of phosphate, glutamate, aspartate, and succinate, and wherein the formulation is stable and retains its biological activity.

In another embodiment, the invention discloses an aqueous pharmaceutical formulation for a therapeutic antibody comprising a dual buffer system, wherein individual buffer in the dual buffer system are selected from the group consisting of phosphate, glutamate, aspartate, and succinate, and wherein the formulation is stable at 2-8° C. for at least 2 years or at 25° C. for at least 3 months or at about 40° C. for at least 2 weeks, or at about 50° C. for at least 1 week, and the formulation inhibits the reduction in monomer content of the antibody composition.

Another embodiment of the invention discloses an aqueous pharmaceutical formulation comprising a therapeutic antibody and a dual buffer system, wherein individual buffer in the dual buffer system are selected from the group consisting of phosphate, glutamate, aspartate, and succinate, and wherein the formulation is stable at 2-8° C. for at least 2 years or at 25° C. for at least 3 months or at about 40° C. for at least 2 weeks, or at about 50° C. for at least 1 week, and the formulation inhibits the reduction in main peak content of the antibody composition.

In an embodiment, the invention discloses an aqueous pharmaceutical formulation comprising a therapeutic antibody and a dual buffer system, wherein individual buffer in the dual buffer system are selected from the group consisting of phosphate, glutamate, aspartate, and succinate, and wherein the percentage recovery of the therapeutic antibody in the dual buffer is increased when compared with the single buffer system.

In yet another embodiment, the invention discloses an aqueous pharmaceutical formulation of adalimumab comprising a phosphate-glutamate dual buffer system, wherein the formulation is stable at 25° C. for 3 months and retains its biological activity.

An embodiment, the invention discloses an aqueous pharmaceutical formulation of adalimumab comprising a dual buffer system selected from phosphate-glutamate buffer or succinate-glutamate buffer, wherein the formulation is stable at 25° C. for 3 months or 37° C. for 4 weeks or 40° C. for 4 weeks or 50° C. for 2 weeks, and wherein the percentage of monomer content is not less than 90%, more preferably not less than 98%.

In another embodiment, the invention discloses an aqueous pharmaceutical formulation of adalimumab comprising a dual buffer system selected from phosphate-glutamate buffer or succinate-glutamate buffer, wherein the formulation is stable at 25° C. for 3 months or 37° C. for 4 weeks or 40° C. for 4 weeks or 50° C. for 2 weeks, and wherein the reduction in monomer content of the antibody composition is less than 7.5%, and more preferably less than 2.5%.

Yet another embodiment, the invention discloses an aqueous pharmaceutical formulation of adalimumab comprising a dual buffer system selected from phosphate-glutamate buffer or succinate-glutamate buffer, wherein the formulation is stable at 25° C. for 3 months or 37° C. for 4 weeks or 40° C. for 4 weeks or 50° C. for 2 weeks, and wherein the percentage reduction in monomer content of the antibody composition is not greater than 10%, and more preferably not greater than 2.5%.

In another embodiment, the invention discloses an aqueous pharmaceutical formulation of adalimumab comprising phosphate-glutamate dual buffer system, wherein the formulation is stable even after subjecting to multiple freeze-thaw cycles. The formulation is stable post three freeze-thaw cycles and/or stable post five freeze-thaw cycles. The concentration of adalimumab herein is about 50 mg/ml to about 100 mg/ml.

An embodiment of the invention discloses, an aqueous pharmaceutical formulation of adalimumab comprising dual buffer system selected from phosphate-glutamate buffer or succinate-glutamate buffer, wherein the formulation is stable at 25° C. for 3 months or 37° C. for 4 weeks or 40° C. for 4 weeks or 50° C. for 2 weeks, and wherein the percentage of main peak of the antibody composition is greater than 35%, and preferably greater than 65%.

In another embodiment of the invention discloses, an aqueous pharmaceutical formulation of adalimumab comprising a dual buffer system selected from phosphate-glutamate buffer or succinate-glutamate buffer, wherein the formulation is stable at 25° C. for 3 months or 37° C. for 4 weeks or 40° C. for 4 weeks or 50° C. for 2 weeks, and wherein reduction in the main peak of the antibody composition is in the range of about 10-40%.

An embodiment of the invention discloses an aqueous pharmaceutical formulation of adalimumab comprising a dual buffer system selected from phosphate-glutamate buffer or succinate-glutamate buffer, wherein the formulation is stable at 25° C. for 3 months or 37° C. for 4 weeks or 40° C. for 4 weeks or 50° C. for 2 weeks, and wherein the percentage reduction in the main peak of the antibody composition is in the range of about 15-55%.

Another embodiment discloses an aqueous pharmaceutical formulation of tocilizumab comprising succinate-aspartate dual buffer system, wherein the formulation is stable at 40° C. for 2 weeks, and wherein the monomer content of the antibody composition is not less than 95%.

Yet another embodiment, the invention discloses an aqueous pharmaceutical formulation of tocilizumab comprising succinate-aspartate dual buffer system, wherein the formulation is stable at 40° C. for 2 weeks, and wherein the reduction in monomer content of the antibody composition is not more than 2.5%.

Further embodiment, the invention discloses an aqueous pharmaceutical formulation of tocilizumab comprising succinate-aspartate dual buffer system, wherein the formulation is stable at 40° C. for 2 weeks, and wherein the percentage reduction in monomer content of the antibody composition is not more than 2.5%.

In another embodiment, the invention discloses an aqueous pharmaceutical formulation of tocilizumab comprising succinate-aspartate dual buffer system, wherein the formulation is stable at 40° C. for 2 weeks, and wherein the main peak content of the antibody composition is not less than 60%.

Another embodiment of the invention discloses, an aqueous pharmaceutical formulation of tocilizumab comprising succinate-aspartate dual buffer system, wherein the formulation is stable at 40° C. for 2 weeks, and wherein the reduction in main peak content is not more than 5%.

Yet another embodiment, the invention discloses an aqueous pharmaceutical formulation of tocilizumab comprising succinate-aspartate dual buffer system, wherein the formulation is stable at 40° C. for 2 weeks, and wherein the percentage reduction in main peak content of the antibody composition is not more than 10%.

In another embodiment of the invention discloses, an aqueous pharmaceutical formulation of trastuzumab comprising a dual buffer system selected from the group consisting of phosphate-glutamate or succinate-glutamate, and the formulation is stable at 37° C. for 2 weeks or 50° C. for 1 week, and wherein the monomer content of the antibody composition is not less than 95%.

Yet another embodiment, the invention discloses an aqueous pharmaceutical formulation of trastuzumab comprising a dual buffer system selected from the group consisting of phosphate-glutamate and succinate-glutamate, and the formulation is stable at 37° C. for 2 weeks or 50° C. for 1 week, and wherein the reduction in monomer content of the antibody composition is not more than 5.0%.

Further embodiment of the invention discloses, an aqueous pharmaceutical formulation of trastuzumab comprising a dual buffer system selected from the group consisting of phosphate-glutamate and succinate-glutamate, wherein the formulation is stable at 37° C. for 2 weeks or 50° C. for 1 week and inhibits the reduction in monomer content of the antibody composition, wherein the percentage reduction in monomer content is not more than 5.0%.

In an embodiment, the invention discloses an aqueous pharmaceutical formulation of trastuzumab comprising a dual buffer system selected from a group consisting of phosphate-glutamate and succinate-glutamate, wherein the percentage recovery in the final formulation dual buffer is greater than 50%.

### Definitions

The term “antibody” as used herein encompasses whole antibodies and any antigen binding fragment (i.e., “antigen-binding portion”) or single chains or fusion protein thereof. An “antibody” refers to a glycoprotein comprising at least two heavy (H) chains and two light (L) chains inter-connected by disulfide bonds, or an antigen binding portion thereof.

The term “stable” formulation refers to the formulation wherein the antibody therein retains its physical stability and/or chemical stability and/or biological activity upon storage.

Stability studies provides evidence of the quality of an antibody under the influence of various environmental factors during the course of time. ICH's “Q1A: Stability Testing of New Drug Substances and Products,” states that data from accelerated stability studies can be used to evaluate the effect of short-term excursions higher or lower than label storage conditions that may occur during the shipping of the antibodies.

The term ‘freeze-thaw cycle’ as used herein describes a process of freezing a drug substance or drug product to lower temperatures such as −50° C. or even lower temperatures such as −80 C and followed by thawing at room temperature.

Various analytical methods are available for measuring the physical and chemical degradation of the antibody in the pharmaceutical formulations. An antibody “retains its physical stability” in a pharmaceutical formulation if it shows substantially no signs of aggregation, precipitation and/or denaturation upon visual examination of color and/or clarity, or as measured by UV light scattering or by size exclusion chromatography. An antibody is said to “retain its chemical stability” in a pharmaceutical formulation when its shows no or minimal formation of product variants which may include variants as a result of chemical modification of antibody of interest such as deamination, oxidation etc. Analytical methods such as ion exchange chromatography and hydrophobic ion chromatography may be used to investigate the chemical product variants.

The term ‘monomer’ as used herein describes antibodies consisting of two light chains and two heavy chains. The monomer content of an antibody composition is typically analyzed by size exclusion chromatography (SEC). As per the separation principle of SEC the large molecules or molecules with high molecular weight (HMW) elute first followed by smaller or lower weight molecules. In a typical SEC profile for an antibody composition, aggregates that may include dimers, multimers, etc., elute first, followed by monomer, and the clipped antibody variants or degradants may be eluted last. In some circumstances the aggregate peak or the degradant peaks may not elute as a baseline separated peaks but instead as a shoulder or abnormal broad peaks. In order to maintain the appropriate activity of an antibody, in particular of a therapeutic antibody, it is desirable to reduce the formation of aggregate or degradant products and hence control the monomer content to a target value. Ability to inhibit the formation of aggregate and degradant content as measured at various time points during stability studies may indicate the suitability of the candidate formulation for antibody of interest. TSK-GEL G3000SWXL (7.8 mm×30 cm) column from TOSCH can be used on water HPLC to perform SEC.

The term ‘main peak’ as used herein refers to the peak that elutes in abundance (major peak) during a cation exchange chromatography. The peak that elutes earlier than the main peak, during a cation exchange chromatography, with a charge that is acidic relative to the main peak is termed acidic variant peak. The peak that elutes later than the main peak, during a cation exchange chromatography, with a charge that is relatively basic than the main peak is termed as basic variant peak. The main peak content can be determined by Ion exchange chromatography (IEC). There are two modes of IEC available cation and anion exchange chromatography. Positively charged molecules bind to anion exchange resins while negatively charged molecules bind to cation exchange resins. In a typical cation exchange chromatographic profile of an antibody composition acidic variants elute first followed by the main peak) and thereafter lastly the basic variants will be eluted. The acidic variants are a result of antibody modifications such as deamidation of asparagine residues. The basic variants are a result of incomplete removal of C-terminal lysine residue(s). In general, in an antibody a lysine residue is present at the C-terminal end of both heavy and light chain. An antibody molecule containing lysine at both heavy and light chain is referred to as K2 variant, the antibody molecule containing lysine residue at either one of heavy and light chain is referred to as K1 variant and antibody molecule having none is KO molecule. Carboxypeptidase B (CP-B enzyme) enzyme acts on the C-terminal lysine residues present on K2 and K1 variants and thus converting them as KO molecules. As per circumstances of the case, the IEC analysis can be carried out for samples digested with carboxypeptidase B (CP-B) enzyme. In a typical stability study it is expected that a stable formulation leads to reduction in formation of chargevariants (acidic and basic variants), during the study, and hence minimize any reduction in main peak content.

An antibody “retains its biological activity” in a pharmaceutical formulation, if the antibody is biologically functional to perform its intended purpose. For example, the biological activity of an antibody can be determined by in vitro cell based assays such as antigen binding/neutralization assays, in case of anti-TNF antibody, biological activity is determined by a TNF a cytotoxicity neutralization assay.

The term “percentage recovery” refers to the proportion of the antibody concentration obtained in the final formulation buffer to the antibody concentration in the process buffer which precedes the formulation step for example the last downstream process elution buffer.

The high concentration formulation for an antibody refers to a formulation, which enables higher dose to be administered to a subject using a volume, which is equal to, or less than the formulation for standard treatment.

Pharmaceutically acceptable excipients refer to the additives or carriers, which may contribute to stability of the antibody in formulation. The excipients may encompass stabilizers and tonicity modifiers. Examples of stabilizers and tonicity modifiers include but not limited to sugars, amino acids, polyols, salts or surfactants, and derivatives and combination thereof.

Sugars and polyols can be referred to monosaccharides, disaccharide, and polysaccharides. Examples of sugars include, but are not limited to, sucrose, glucose, dextrose, and others. Additionally, polyol refers to an alcohol containing multiple hydroxyl groups. Examples of polyols include, but are not limited to, mannitol, sorbitol, and others.

Surfactant refers to pharmaceutically acceptable excipients used to protect the protein formulations against various stress conditions, like agitation, shearing, exposure to high temperature etc. The suitable surfactants include but are not limited to polyoxyethylensorbitan fatty acid esters such as Tween 20™ or Tween 80™, polyoxyethylene-polyoxypropylene copolymer (e.g. Poloxamer, Pluronic), sodium dodecyl sulphate (SDS) and the like or combination thereof.

Salts are used as tonicity modifiers and examples of salts include but not limited to sodium chloride, potassium chloride, magnesium chloride, arginine hydrochloride, sodium thiocyanate, ammonium thiocyanate, ammonium sulfate, ammonium chloride, calcium chloride, zinc chloride and/or sodium acetate.

One or more amino acids may also be part of an antibody formulation and can be selected from a basic amino acids or hydrophobic amino acids or a combination thereof. The basic amino acid can be selected from the group consisting of arginine, lysine, histidine and their salts or derivatives thereof whereas hydrophobic amino acid can be selected from the group consisting of glycine, alanine, valine, leucine, phenyl alanine, methionine, tryptophan and their salts or derivatives thereof.

Certain specific aspects and embodiments of the invention are more fully described by reference to the following examples. However, these examples should not be construed as limiting the scope of the invention in any manner.

### EXAMPLES

To achieve a stable aqueous formulation for an antibody, as part of experimental design, various dual buffers as disclosed in the present invention were evaluated. Different dual buffers were assessed for a range of therapeutic antibodies. The stable formulation may further comprises pharmaceutically acceptable excipients. The stability of the formulations of antibodies in the dual buffer system was estimated at real time as well as under accelerated conditions. The stability of the antibodies in the dual buffer formulation was investigated by analytical assays for any chemical and physical degradation. This formulation of antibodies in dual buffer system is particularly advantageous for high concentration antibodies.

### Example 1: Single Buffer Vs. Dual Buffer System

50 mg/ml of Adalimumab (A-mab) was formulated in single buffer systems and dual buffer systems. The details of single and dual buffer system are as given in Table-1.

TABLE 1

| Formulation Composition | Final pH |
|---|---|
| 20 mM Glutamic Acid | 5.20 |
| 19.20 mM Phosphate buffer | 5.23 |
| 23 mM Phosphate-Glutamate Buffer | 5.25 |
| 20 mM Phosphate-Citrate Buffer | 5.21 |

All of the above formulations further comprise polysorbate 80 in the final concentration of 0.1%.

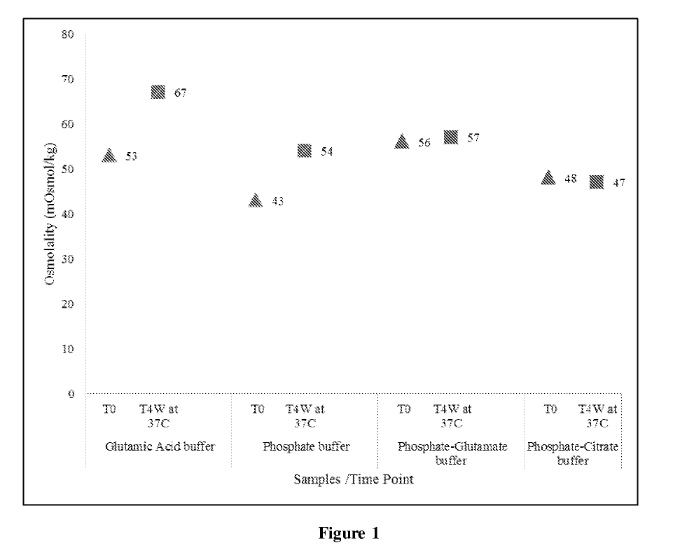

The above formulations of adalimumab were evaluated for stability at 37° C. for 4 weeks and thereafter analyzed for monomer content by size exclusion chromatography and also main peak content using ion exchange chromatography and the results of the same are given in Table 2 & 3. T0 in table represents the monomer/main peak content at starting time point. Also, the osmolality shift was observed for formulations as disclosed in FIG. 1.

TABLE 2

| | | % High | | % | % Low |
|---|---|---|---|---|---|
| Formulation | Time | molecular | % | Monomer | Molecular |
| Composition | Point | weight | Monomer | Shoulder | weight |
| A-mab in | T0 | 0.6 | 99.4 | ND | 0.0 |
| Glutamic Acid | T4W at | 3.9 | 92.9 | 2.2 | 1.03 |
| buffer | 37° C. | | | | |
| A-mab in | T0 | 0.7 | 99.3 | ND | 0.0 |
| Phosphate | T4W at | 1.4 | 93.8 | 3.1 | 1.7 |
| buffer | 37° C. | | | | |
| A-mab in | T0 | 0.7 | 99.3 | ND | 0.0 |
| Phosphate | T4W at | 2.1 | 97.4 | ND | 0.6 |
| Glutamate | 37° C. | | | | |
| buffer | | | | | |
| A-mab in | T0 | 0.7 | 99.2 | ND | 0.1 |
| Phosphate | T4W at | 2.2 | 97.3 | ND | 0.5 |
| Citrate buffer | 37° C. | | | | |

ND—Not detected

TABLE 3

| Formulation | | % Acidic | % Main | % Basic |
|---|---|---|---|---|
| Composition | Time Point | peak | peak | peak |
| A-mab in | T0 | 23.9 | 74.8 | 1.2 |
| Glutamic Acid | T4W at 37° C. | 68.4 | 28.0 | 3.6 |
| buffer | | | | |
| A-mab in | T0 | 25.9 | 72.3 | 1.8 |
| Phosphate buffer | T4W at 37° C. | 45.9 | 46.5 | 7.6 |
| A-mab in | T0 | 24.2 | 74.3 | 1.6 |
| Phosphate | T4W at 37° C. | 46.4 | 50.0 | 3.6 |
| Glutamate buffer | | | | |
| A-mab in | T0 | 23.3 | 74.9 | 1.8 |
| Phosphate Citrate | T4W at 37° C. | 49.5 | 47.3 | 3.2 |
| buffer | | | | |

As can be noted from table 2 and table 3 that the reduction in the monomer content as well as main peak content is minimum in A-mab formulated in phosphate glutamate buffer as compared to A-mab in single buffer systems i.e. phosphate or glutamic acid buffer as well as A-mab in other dual buffer system i.e. citrate and phosphate dual buffer system. FIG. 1 represents the osmolality shift at T0 and T4 weeks at 37° C. for A-mab in formulations disclosed in Table-1.

### Example 2: Formulation of Adalimumab in Different Dual Buffers & Excipients

50 mg/ml of adalimumab was formulated in two different dual buffer combinations containing various concentration and combination of excipients as detailed in table 4. Further, same concentration of adalimumab was formulated in citrate-phosphate dual buffer as a control.

TABLE 4

| Formulations ID | Formulation Composition |
|---|---|
| Formulation-1 | 50 mg/ml adalimumab |
| (Control_50 mg/ml)- | 20 mM Citrate-Phosphate buffer |
| (F1) | 1.2% mannitol |
| | 105.45 mM NaCl |
| | 0.1% Polysorbate-80, |
| | pH 5.2 |
| Formulation-2 (F2) | 50 mg/ml adalimumab |
| | 23 mM Phosphate-glutamate buffer |
| | 50 mg/ml of sorbitol |
| | 15 mM NaCl |
| | 0.1% Polysorbate-80 |
| | pH 5.2 |
| Formulation-3 (F3) | 50 mg/ml adalimumab |
| | 23 mM Phosphate-glutamate buffer |
| | 80 mg/ml of sucrose |
| | 15 mM NaCl |
| | 0.1% Polysorbate-80 |
| | pH 5.2 |
| Formulation-4 (F4) | 50 mg/ml adalimumab |
| | 23 mM Succinate-glutamate buffer |
| | 80 mg/ml of sucrose |
| | 15 mM NaCl |
| | 0.1% Polysorbate-80 |
| | pH 5.2 |
| Formulation-4 (F5) | 50 mg/ml adalimumab |
| | 23 mM Succinate-glutamate buffer |
| | 80 mg/ml of trehalose |
| | 15 mM NaCl |
| | 0.1% Polysorbate-80 |
| | pH 5.2 |

The above formulations of adalimumab evaluated for stability at 50° C. for 2 weeks and F2 and F4 were also evaluated for stability at 25° C. for 3 months. Furthermore, F2 was also evaluated for stability at 37° C. for 4 weeks.

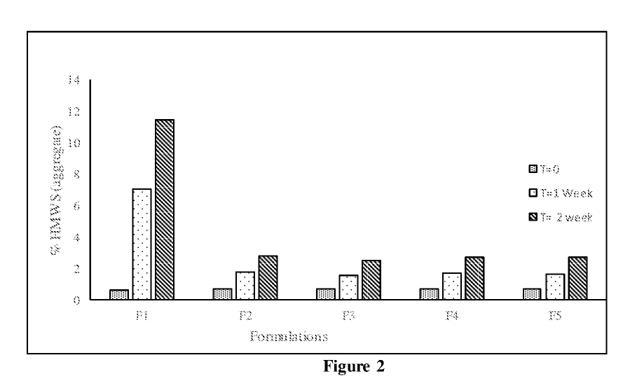

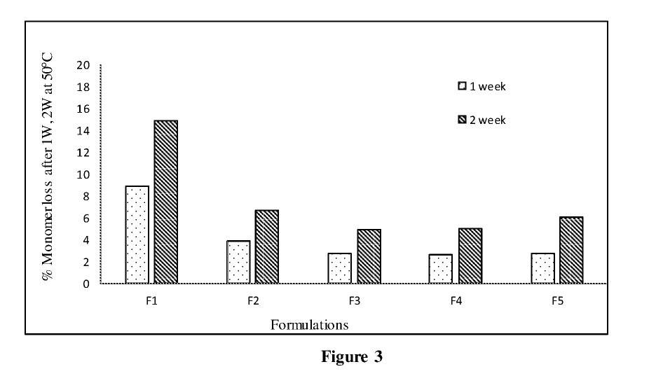

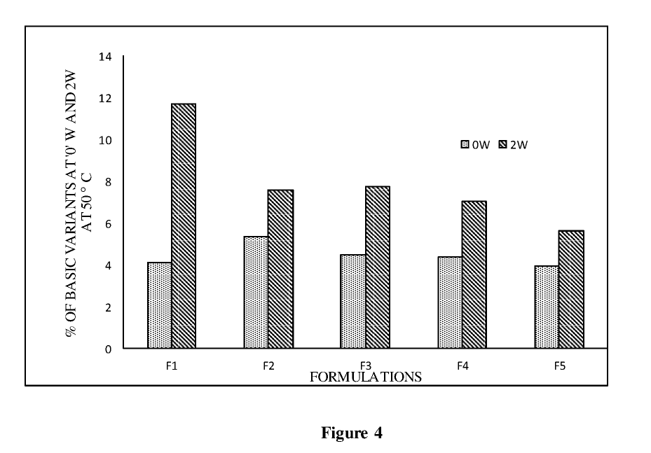

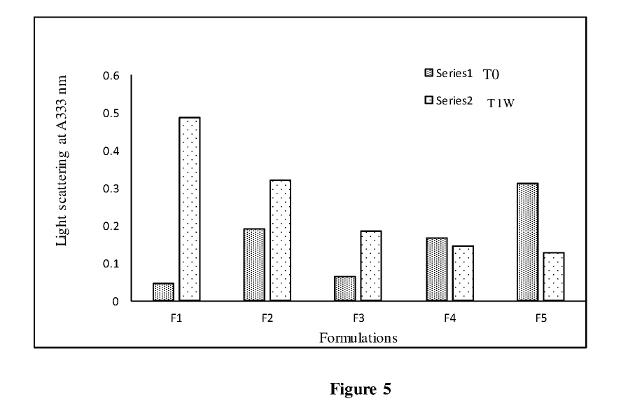

The formulation were thereafter analyzed for monomer content by size exclusion chromatography and also main peak content using ion exchange chromatography and results are given in Table 5 to 11. T0 in table represents the monomer/main peak content at starting time point. FIG. 2 demonstrates % of high molecular weight species (HMWS)/aggregate content for formulations F1-F5 stored at 50° C. for T0, T1 (week 1) and T2 (week 2). FIG. 3 demonstrates % of monomer loss for formulations F1-F5 stored at 50° C. at 1 week and 2 week. FIG. 4 demonstrates % of basic variants for formulations F1-F5 stored at 50° C. for 0W and 2W (2 weeks). FIG. 5 demonstrates light scattering data for formulations F1-F5 stored at 50° C. for T0 and T1W (1 weeks).

TABLE 5

| | | Average % | |
|---|---|---|---|
| | | Relative | Standard |
| Formulation ID | Time point | Potency | Deviation |
| F1 | T0 | 92.0 | 5.8 |
| | T3 month@ 25° C. | 99.4 | 4.9 |
| F2 | T0 | 98.6 | 4.9 |
| | T3 month@ 25° C. | 91.7 | 2.5 |
| F4 | T0 | 93.6 | 5.0 |
| | T3 month@ 25° C. | 100.4 | 2.3 |

TABLE 6

Monomer Peak

| Formulation ID | T0 | T2 weeks at 50° C. |
|---|---|---|
| Formulation-1 (Control)-F1 | 97.3 | 82.3 |
| Formulation-2 (F2) | 97.0 | 89.7 |
| Formulation-3 (F3) | 97.1 | 92.2 |
| Formulation-4 (F4) | 97.0 | 92.1 |
| Formulation-4 (F5) | 97.1 | 91.1 |

TABLE 7

Main Peak

| | | T2 weeks at |
|---|---|---|
| Formulation ID | T0 | 50° C. |
| Formulation-1 (Control)-F1 | 75.4 | 36.9 |
| Formulation-2 (F2) | 76.0 | 36.1 |
| Formulation-3 (F3) | 75.9 | 37.8 |
| Formulation-4 (F4) | 76.8 | 35.5 |
| Formulation-4 (F5) | 77.0 | 34.0 |

TABLE 8

Monomer Peak

| | | T3 months at |
|---|---|---|
| Formulation ID | T0 | 25° C. |
| Formulation-1 (Control)-F1 | 99.1 | 96.9 |
| Formulation-2 (F2) | 99.1 | 97.3 |
| Formulation-4 (F4) | 99.2 | 97.0 |

TABLE 9

Main Peak

| | | T3 months at |
|---|---|---|
| Formulation ID | T0 | 25° C. |
| Formulation-1 (Control)-F1 | 73.8 | 63.5 |
| Formulation-2 (F2) | 73.5 | 63.5 |
| Formulation-4 (F4) | 73.1 | 53.1 |

TABLE 10

Monomer Peak

| | | T4 weeks at |
|---|---|---|
| Formulation ID | T0 | 37° C. |
| Formulation-1 (Control)-F1 | 99.1 | 95.9 |
| Formulation-2 (F2) | 99.1 | 96.5 |

TABLE 11

Main Peak

| | | T4 weeks at |
|---|---|---|
| Formulation ID | T0 | 37° C. |
| Formulation-1 (Control)-F1 | 73.8 | 54.8 |
| Formulation-2 (F2) | 73.5 | 55.4 |

Furthermore, F1-F5 samples were subjected for multiple freeze-thaw cycles by freezing the said samples to −80° C. using a deep freezer and thawed at room temperature. These freeze-thaw cycles were repeated for five times then the samples were subjected for SEC and IEC to check effect of freeze-thaw cycles on monomer content and main peak of the adalimumab 50 mg/ml respectively and results of the same has been given in table 12 and table 13.

TABLE 12

Monomer Peak

| Formulation ID | 0 X | 1 X | 3X | 5X |
|---|---|---|---|---|
| Control (50 mg/ml)- | 98.65 | 98.68 | 98.68 | 98.73 |
| F1 | | | | |
| F2 | 98.67 | 98.73 | 98.77 | 98.77 |
| F3 | 98.68 | 98.70 | 98.74 | 98.73 |
| F4 | 98.69 | 98.73 | 98.75 | 98.75 |
| F5 | 98.73 | 98.73 | 98.76 | 98.76 |

X-indicates number of freeze-thaw cycles

TABLE 13

Main Peak

| Formulation ID | 0 X | 5X |
|---|---|---|
| Control (50 mg/ml)- | 60.1 | 61.8 |
| F1 | | |
| F2 | 61.19 | 59.95 |
| F3 | 63.15 | 60.23 |
| F4 | 61.46 | 60.44 |
| F5 | 60.62 | 60.66 |

X-indicates number of freeze-thaw cycles.

50 mg/ml of adalimumab was formulated in F6 and F7 buffers, the composition of the same is disclosed in Table 14.

TABLE 14

| Formulations ID | Formulation Composition |
|---|---|
| Formulation-6 | 50 mg/ml adalimumab |
| (F6) | 23 mM Phosphate-Glutamate buffer |
| | 30 mg/ml Mannitol |
| | 30 mM Arginine |
| | 10 mM Glycine |
| | 15 mM Sodium chloride |
| | pH 5.2 |
| Formulation-6 | 50 mg/ml adalimumab |
| (F7) | 23 mM Phosphate-Glutamate buffer |
| | 30 mg/ml Mannitol |
| | 50 mM Sodium chloride |
| | pH 5.2 |

The above formulations of adalimumab in F6 and F7 were evaluated for stability at 40° C. for 4 weeks. F1, F6 and F7 samples stored at 25° C. for 3 months were also subjected for evaluation of their biological activity using standard assay procedure of TNF-α cytotoxicity neutralization assay wherein L929 cells expressing TNF receptors were used and average relative potency of the samples were calculated and results are represented in table 15.

TABLE 15

| | | Average % | |
|---|---|---|---|
| | | Relative | Standard |
| Formulation ID | Time point | Potency | Deviation |
| F1 | T0 | 92.0 | 5.8 |
| | T3 month@ 25° C. | 99.4 | 4.9 |
| F6 | T0 | 98.6 | 4.9 |
| | T3 month@ 25° C. | 91.7 | 2.5 |
| F7 | T0 | 93.6 | 5.0 |
| | T3 month@ 25° C. | 100.4 | 2.3 |

The formulation were thereafter analyzed for monomer content by size exclusion chromatography and also main peak content using ion exchange chromatography and results are given in Table 16 and 17 respectively. The data in Table 14 is for samples which had not been treated with carboxypeptidase-B.

TABLE 16

Monomer Peak

| | | T4 weeks at |
|---|---|---|
| Formulation ID | T0 | 40° C. |
| F6 | 99.1 | 96.3 |
| F7 | 99.0 | 95.7 |

TABLE 17

Main Peak

| | | T4 weeks at |
|---|---|---|
| Formulation ID | T0 | 40° C. |
| F6 | 48.6 | 39.7 |
| F7 | 49.3 | 39.3 |

From, the above results it is evident that, 50 mg/ml of adalimumab formulated in a dual buffer systems such as phosphate-glutamate buffer and succinate-glutamate buffer exhibits stability even under accelerated stability conditions.

### Example 3: High Concentration Antibody Formulations

100 mg/ml of adalimumab was formulated in 20 mM phosphate-glutamate buffer system and further comprising other pharmaceutical acceptable excipients as represented in table 18. Further, pharmaceutically acceptable excipients were added in following concentration/concentration ranges such as arginine at a concentration range of about 40 to 120 mM; glycine at a concentration of about 50 mM and polyols at a concentration of 5-10 mg/ml and Nacl at a concentration of 5-10 mM. 100 mg/ml of adalimumab approved formulation is used as control in this experiment.

TABLE 18

| Formulation ID | Formulation Composition |
|---|---|
| Control (100 | 100 mg/ml A-mab, 42 mg/mL Mannitol, 0.1% |
| mg/ml)_F8 | Polysorbate 80 |
| F9 | 100 mg/ml A-mab, Phosphate glutamate buffer, |
| | Glycine, Arginine, NaCl, 0.1% Polysorbate 80 |
| F10 | 100 mg/ml A-mab, Phosphate glutamate buffer, |
| | Glycine, Arginine, Mannitol |
| F11 | 100 mg/ml A-mab, Phosphate glutamate buffer, |
| | Arginine, 0.1% Polysorbate 80 |
| F12 | 100 mg/ml A-mab, Phosphate glutamate buffer, |
| | Glycine, Arginine, Sorbitol |

The above formulations of adalimumab were evaluated for stability at 40° C. for 4 weeks. The formulations were thereafter analyzed for monomer content by size exclusion chromatography and also main peak content using ion exchange chromatography and results are as given in Table 17 & 18. T0 in table represents the monomer/main peak content at starting time point. The data in Table 17 is for samples which had not been treated with carboxypeptidase-B. F8, F9 and F11 samples were further subjected for multiple freeze-thaw cycles by freezing the said samples to −80° C. using a deep freezer and thawed at room temperature. These freeze-thaw cycles were repeated for five times then the samples were checked for particulate matter by visual inspection and results are given in table 19. Further, the samples were subjected for SEC to check effect of freeze-thaw cycles on monomer content of the adalimumab 100 mg/ml and results of the same has been given in table 20.

TABLE 19

Freeze-Thaw Cycles

| Formulation ID | 0 X | 1 X | 3X | 5X |
|---|---|---|---|---|
| Control (100 | Clear | Clear | Clear | Clear |
| mg/ml)-F8 | | | | |
| F9 | Clear | Clear | Clear | Clear |
| F11 | Clear | Clear | Clear | Clear |

X-indicates number of freeze-thaw cycles

TABLE 20

Monomer Peak

| Formulation ID | 0 X | 1 X | 3X | 5X |
|---|---|---|---|---|
| Control (100 | 98.4 | 98.4 | 98.4 | 98.4 |
| mg/ml)-F8 | | | | |
| F9 | 99.0 | 99.0 | 99.0 | 99.1 |
| F11 | 99.1 | 99.1 | 99.0 | 99.0 |

X-indicates number of freeze-thaw cycles

TABLE 21

Monomer Peak

| | | T4 weeks at |
|---|---|---|
| Formulation ID | T0 | 40° C. |
| Control (100 mg/ml)-F8 | 98.2 | 88.0 |
| F9 | 98.6 | 94.9 |
| F10 | 98.7 | 94.2 |
| F11 | 98.8 | 94.2 |
| F12 | 98.7 | 93.7 |

TABLE 22

Main Peak

| | | T4 weeks at |
|---|---|---|
| Formulation ID | T0 | 40° C. |
| Control (100 mg/ml)-F8 | 77.2 | 52.5 |
| F9 | 55.8 | 49.1 |
| F10 | 56.1 | 46.5 |
| F11 | 54.5 | 44.9 |
| F12 | 56.2 | 49.6 |

From the above results, it is very evident that 100 mg/ml of adalimumab formulated in a dual buffer system such as phosphate-glutamate buffer exhibits better stability under accelerated conditions as compared to the approved 100 mg/ml of adalimumab formulation.

### Example 4: Other Antibodies Formulated in Dual Buffer System

180 mg/ml of tocilizumab (Toc-mab) was formulated in a dual buffer system comprising Succinate-Aspartate buffer system as represented in below table 23. Approved 180 mg/ml of tocilizumab is formulated in histidine buffer and the same had been used as control in this experiment.

TABLE 23

| Formulation ID | Formulation Composition |
|---|---|
| Toc- (Control) | 180 mg/ml Toc-mab, 20 mM L-Histidine-L-Histidine |
| | monohydrochloride monohydrate |
| Toc -1 | 180 mg/ml Toc-mab, 20 mM Succinate-Aspartate buffer |

The above formulations of tocilizumab were stored at 40° C. for 2 weeks. The formulations were thereafter analyzed for monomer content by size exclusion chromatography and also main peak content using ion exchange chromatography and results are given in Table 24 & 25. T0 in table represents the monomer/main peak content at starting time point.

TABLE 24

Monomer Peak

| | | T2 weeks at |
|---|---|---|
| Formulation ID | T0 | 40° C. |
| Toc (Control) | 98.0 | 96.1 |
| Toc 1 | 97.7 | 95.4 |

TABLE 25

Main Peak

| | | T2 weeks at |
|---|---|---|
| Formulation ID | T0 | 40° C. |
| Toc (Control) | 66.2 | 58.3 |
| Toc 1 | 64.0 | 59.9 |

From the above results, it is evident that 180 mg/ml of tocilizumab formulated in a dual buffer system such as succinate-aspartate provides equivalent stability as that of tocilizumab formulated in histidine buffer under accelerated conditions.

Further, as part of evaluating dual buffer system for different therapeutic antibodies, 21 mg/ml of trastuzumab (T-mab) was formulated in a buffer system comprising a dual buffer systems such as phosphate-glutamate buffer and succinate-glutamate buffer which further comprises pharmaceutically acceptable excipients as represented in Table 26 below. Further, the pharmaceutically acceptable excipients were added in following concentration ranges in trastuzumab, trehalose at a concentration of about 150 mM, more specifically 200 mM; methionine at a concentration of 5 mM; Arginine at a concentration of about 25 mM; sucrose and sorbitol at a concentration range of 1.2 to 2% and 0.04 mg/ml of polysorbate 20.

Prior to the formulation of trastuzumab in the dual buffer systems, buffer exchange step has been performed to transfer the trastuzumab drug substance obtained from downstream process present in a different buffer background to the said dual buffer and percentage recovery of the same has been calculated and represented in table 27.

TABLE 26

| | | | pH |
|---|---|---|---|
| Formulation | | | of the |
| ID | Buffer Composition | Excipients | buffer |
| T- | 20 mM Histidine, | Trehalose, methionine, | 5.5 |
| mab_Control | Histidine HCl buffer | polysorbate 20, | |
| T-mab-1 | 20 mM Phosphate- | Trehalose, methionine, | 6.0 |
| | Glutamate | polysorbate 20 | |
| T-mab-2 | 20 mM Succinate- | Trehalose, methionine, | 5.2 |
| | Glutamate | polysorbate 20 | |
| T-mab-3 | 20 mM Succinate- | Trehalose, methionine, | 6.0 |
| | Glutamate | polysorbate 20 | |
| T-mab-4 | 20 mM Phosphate- | Sucrose, Arginine•HCl, | 5.5 |
| | Glutamate | methionine, polysorbate | |
| | | 20 | |
| T-mab-5 | 20 mM Phosphate - | Sorbitol, Arginine•HCl, | 5.5 |
| | Glutamate | methionine, Polysorbate | |
| | | 20 | |

TABLE 27

| Formulation ID | % Recovery |
|---|---|
| T-mab_Control | 49.6 |
| T-mab_1 | 59.9 |
| T-mab_2 | 73.9 |
| T-mab_3 | 62.8 |
| T-mab_4 | 51.1 |
| T-mab_5 | 65.8 |

The above formulations of trastuzumab were subjected for accelerated stability studies by keeping the said samples at 37° C. for 2 weeks and 50° C. for 1 week. The formulations were thereafter analyzed for monomer content by size exclusion and results are given in Table 28. T0 in table represents the monomer/main peak content at starting time point.

TABLE 28

| | Monomer Peak | | |
|---|---|---|---|
| | | T2 weeks at | T1 weeks at |
| Formulation ID | T0 | 37° C. | 50° C. |
| T-mab_Control | 99.6 | 95.9 | 96.8 |
| T-mab_1 | 99.6 | 98.8 | 97.1 |
| T-mab_2 | 99.6 | 97.8 | 95.9 |
| T-mab_3 | 99.6 | 96.6 | 95.6 |
| T-mab_4 | 99.6 | 96.9 | 96.5 |
| T-mab_5 | 99.6 | 96.5 | 96.6 |

1) A stable aqueous pharmaceutical formulation for an antibody comprising a dual buffer system comprising buffers selected from the group consisting of phosphate, aspartate, succinate, and glutamate.

2) The formulation of claim 1, wherein the formulation further comprises pharmaceutically acceptable excipients such as amino acids, sugars, polyols, salts or surfactants.

3) The formulation of claim 1, wherein the antibody is a therapeutic antibody.

4) The formulation of claim 1, wherein the antibody is stable under at least one of the following accelerated stability conditions such as 25° C. for 3 months or 37° C. for 4 weeks or 40° C. for 2 weeks or 50° C. for 1 week.

5) The formulation of claim 1, wherein the antibody is stable when subjected to multiple freeze-thaw cycles.

6) A stable aqueous pharmaceutical formulation of adalimumab comprising a dual buffer selected from the group consisting of phosphate-glutamate buffer or succinate-glutamate buffer.

7) (canceled)

8) A stable aqueous pharmaceutical formulation of tocilizumab comprising succinate-aspartate buffer, and wherein the formulation inhibits a reduction in monomer content and main peak content of the antibody composition.

9) A stable aqueous pharmaceutical formulation of trastuzumab comprising a dual buffer system selected from succinate-glutamate or phosphate glutamate buffer, and wherein the formulation inhibits a reduction in monomer content of the antibody composition.

10) The formulation according to claim 1, wherein the dual buffer system has a pH between 5-7.

11) The formulation according to claim 1, wherein the antibody concentration is at least 20 mg/ml.

12) The formulation according to claim 1, wherein the antibody concentration is at least 100 mg/ml.

13) The formulation according to claim 6, wherein the dual buffer system has a pH between 5-7.

14) The formulation according to claim 6, wherein the antibody concentration is at least 20 mg/ml.

15) The formulation according to claim 6, wherein the antibody concentration is at least 100 mg/ml.

16) The formulation according to claim 6, wherein the formulation inhibits a reduction in monomer content and main peak content of the antibody composition.

17) The formulation according to claim 6, wherein the formulation is stable when subjected to at least three freeze-thaw cycles.

18) The formulation according to claim 8, wherein the antibody concentration is at least 20 mg/ml.

19) The formulation according to claim 8, wherein the antibody concentration is at least 100 mg/ml.

20) The formulation according to claim 9, wherein the antibody concentration is at least 20 mg/ml.

21) The formulation according to claim 9, wherein the antibody concentration is at least 100 mg/ml.
